(19) United States
(12) Patent Application Publication
(10) Pub. No.: US 2009/0171245 A1
(43) Pub. Date: Jul. 2, 2009

(54) SWAB EXTRACTION DEVICE AND METHODS OF USING

(76) Inventors: James R. Uhl, Rochester, MN (US); Franklin R. Cockerill III, Rochester, MN (US); Christian Aichinger, Munchen (DE)

Correspondence Address: FISH & RICHARDSON P.C., PO BOX 1022, MINNEAPOLIS, MN, 55440-1022, US

(21) Appl. No.: 12/299,405

(22) PCT Filed: May 3, 2007
(86) PCT No.: PCT/US2007/068186

§ 371 Date: Feb. 23, 2009

Related U.S. Application Data
(60) Provisional application No. 60/797,395, filed on May 3, 2006.

(51) Int. Cl. A61B 10/02; A61B 5/00; C12M 1/33

(52) U.S. Cl. 600/572; 600/573; 435/306.1

(57) ABSTRACT

This disclosure describes a swab extraction device that can be used to collect a medical specimen, transport the specimen to a laboratory, and process and analyze the specimen.

### TECHNICAL FIELD

This invention relates to device for collecting a biological sample, and more particularly to a swab extraction device.

### BACKGROUND

The first step of many medical diagnostic procedures involves the use of a swab to obtain a biological sample from a patient. Generally, the biological sample is removed from the swab (e.g., using a liquid buffer) and placed into a tube or vial which, for example, is sent to a clinical laboratory for analysis. Oftentimes, the clinical laboratory will transfer the biological sample from the tube or vial to a different tube prior to analyzing the biological sample. Current procedures allow many opportunities for the biological sample to be lost, contaminated, or mixed up with one or more other samples. In addition, current devices and current collection, transport and analysis procedures require a number of physical manipulations (e.g., with the tube or with the sample itself), thereby resulting in a high prevalence of repetitive motion disease in clinical laboratory employees. Therefore, an improved swab device is necessary that reduces the likelihood of contamination or error in a clinical laboratory setting, and that makes the collection, transport and analysis procedures more ergonomical.

### SUMMARY

This disclosure describes a swab extraction device that can be used to collect a medical specimen, transport the specimen to a laboratory, and process and analyze the specimen. A device within which a specimen can be collected, transported and analyzed reduces or eliminates the potential for contamination of the sample and can significantly reduce the repetitive motion disease associated with processing a large number of specimens on a daily basis.

In one aspect, the invention provides a swab extraction device. Such a device typically includes a swab portion, a swab transport/extraction portion, and a receiving tube portion. Generally, a swab portion includes a handle, a swab stem, and a swab end. Generally, a swab transport/extraction portion includes a swab chamber that removeably houses the swab end and a lysis chamber that includes lysis reagent. Typically, the receiving tube portion is in fluid communication with the swab transport/extraction portion.

Such a device also can include one or more filters positioned, for example, between the swab chamber and the lysis chamber, or between the lysis chamber and the receiving tube portion. In some aspects of the invention, the receiving tube portion is removeable from the device (e.g., from the swab transport/extraction portion).

In another aspect, the invention provides for methods of using a swab extraction device as disclosed herein. For example, a user can obtain a biological sample using the swab portion, replace the swab portion in the device (e.g., in the swab chamber), and send the device (or the receiving tube portion thereof) to a laboratory for analysis. A user in a laboratory would receive the device or the receiving tube portion thereof, would complete the processing of the sample, if necessary (e.g., lysing, purifying, centrifuging, etc.) and perform the required analysis on the specimen.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a schematic showing one embodiment of a swab extraction device.

Like reference symbols in the various drawings indicate like elements.

### DETAILED DESCRIPTION

This disclosure describes a device that can be used to collect a medical specimen, transport the specimen to a laboratory, and process and analyze the specimen. A device within which a specimen can be collected, transported and analyzed reduces or eliminates the potential for contamination of the sample. Such a device also can significantly reduce the repetitive motion disease that is associated with continuously processing a large number of specimen.

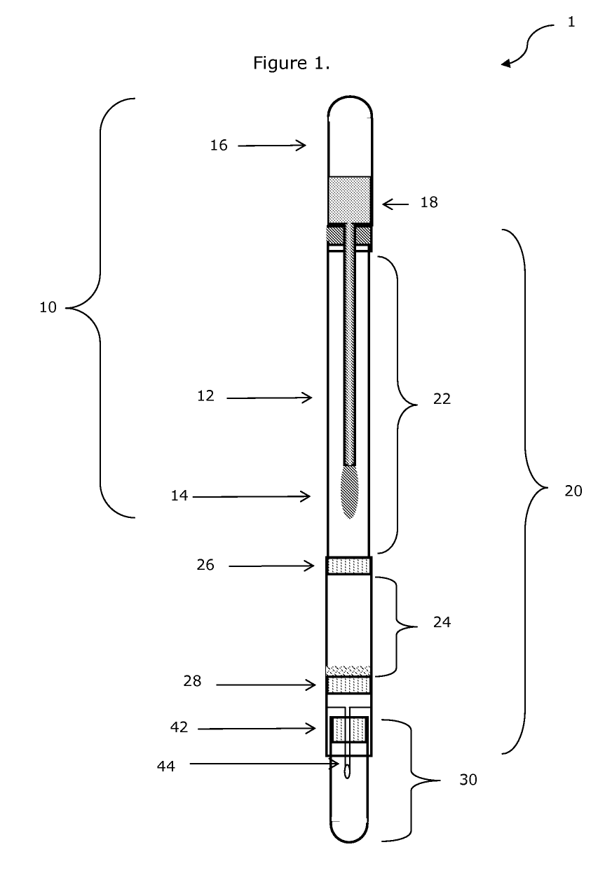

A representative swab extraction device 1 is shown in FIG. 1. A swab extraction device 1 generally includes three portions; a swab portion 10, a swab transport/extraction portion 20, and a receiving tube portion 30. In FIG. 1, the device is in the closed position; that is, the swab portion 10 is contained within the swab transport /extraction portion 20 as it would be during transport of the specimen.

The swab portion 10 of a device generally includes a handle 16 to which one end of the swab stem 12 is attached. The other end of the swab stem 12 contains the swab end 14 for obtaining the specimen. The swab portion can be typical of swabs in the art (see, for example, swab BD-BBL 220116, BD Biosciences, Sparks, M D; and Starswab I I, Starplex Scientific Inc., Etobicoke, Canada). For example, the stem 12 of a swab can be made from a copolymer of polystyrene and butadiene, and the swab end 14 can be made with flocked nylon. In addition, the length of the stem can be, for example, between ten and fifteen cm (e.g., about thirteen cm), but can be shorter or longer depending upon the overall configuration of the device. The swab portion 10 of the device 1 disclosed herein also can include, for example, a chamber 18 containing a liquid carrier for moistening the swab and/or the specimen on the swab. In addition, the liquid carrier can be, for example, a transport buffer or a lysis buffer. As described below, such a buffer could be expelled from the chamber 18 through the swab stem 12 to thereby elute the specimen from the swab end 14.

In FIG. 1, the swab transport/extraction portion 20 includes a swab chamber 22 and a lysis chamber 24. The swab chamber 22 is a cylindrical portion having a lumen into which the swab (stem 12 and end 14) can be removeably placed. The proximal end 23 of the cylindrical portion of the swab transport/extraction portion 20 and the distal end 13 of the swab handle 16 of the swab portion 10 are configured to close securely. Such a closure can include, for example, a snap-fit closure, a screw-type closure, a tapered friction junction, or a pierceable septum. The lysis chamber 24 in the swab transport/extraction portion 20 is a region through which the specimen can travel such that cells in the specimen are lysed. The lysis chamber 24 can contain a lysis reagent, which, for example, can take the form of a liquid buffer (e.g., containing a detergent) or beads that can be used to physically lyse the cells. As disclosed above, a lysis buffer may be contained within a different region of the device and, therefore, a distinct lysis chamber 24 as shown in FIG. 1 is not a required component of the device 1.

The receiving tube portion 30 is the portion of the device into which the specimen (or a portion thereof) ultimately is deposited and analyzed. In some embodiments, the swab transport/extraction portion is integral with the receiving tube portion and both portions (or the entire device) can be used in the analysis. In other embodiments, the receiving tube portion 30, containing the specimen (or a portion thereof), can be removed from the swab transport/extraction portion (e.g., by the user that obtained the specimen, or after transport (e.g., at the laboratory)). In a removable embodiment, the receiving tube portion can be removeably attached to the swab transport/extraction portion by any number of mechanisms such as, for example, a screw-type attachment, a twist-lock attachment, or a pre-scored breakpoint that would allow for the receiving tube portion to be readily separated from the remainder of the device. As one of the goals of the swab extraction device described herein is to reduce the repetitive motions or movements involved in processing a specimen, it is desired that a receiving tube portion 30 be removed from a device 1 as ergonomically as possible.

The device also can contain any of a number of features useful in clinical analysis. For example, a swab extraction device 1 as described herein can include one or more filters 18. The device 1 shown in FIG. 1 includes two filters 18; one is shown positioned between the swab chamber 22 and the lysis chamber 24, and the other is shown positioned between the lysis chamber 24 and the receiving tube portion 30. Such filters can be used to reduce or remove any number of unwanted materials from a biological specimen or materials that might have been introduced into the specimen during processing (e.g., for the purpose of, for example, lysis or sample stabilization). In addition, either or both the swab transport/extraction portion 20 or the receiving tube portion 30 of a swab extraction device 1 can include a label or a place to record information.

The following discloses the use of a representative swab extraction device (e.g., the device shown in FIG. 1). Because a swab extraction device as disclosed herein can have a number of different configurations, however, it is understood by those of skill in the art that the various ways in which a swab extraction device as disclosed herein can be used are not limited to the particular steps described below.

A swab extraction device can be provided to a user (e.g., a nurse or a physician) such that the sterility of the swab, particularly the swab end 14, is maintained. A user, when obtaining a specimen, can grip the swab portion 10 of the device by a handle 16. The swab portion 10 of the device 1 is used to obtain a specimen in the usual manner. The swab portion can be placed into the swab transport/extraction portion 20 and secured by any number of closure means as described herein.

In some embodiments, the entire device can be transported to a laboratory for analysis and, for example, extraction (e.g., lysis) of the specimen takes place during transport. In other embodiments, the receiving tube portion of the device can be removed from the swab transport/extraction portion (after a time sufficient for the specimen to be deposited into the receiving tube portion) and only the receiving tube portion 30 transported to a laboratory for analysis. In still another embodiment, the entire device can be transported to a laboratory and the receiving tube portion removed from the remainder of the device at the laboratory. In yet another embodiment, a device that includes a swab portion 10 and a swab transport/extraction portion 20 and including a biological specimen can be transported to a laboratory and a receiving tube portion 30 added to the device at the laboratory.

‘Extraction’ as used herein generally refers to the different processing steps that might be performed with a biological specimen. For example, ‘extraction’ as used herein can refer to removal of the specimen from the swab end (e.g., elution). ‘Extraction’ also can refer to lysis of the specimen, whether in a formal lysis chamber or elsewhere in the device. ‘Extraction’ also can refer to the process of depositing the specimen (or a portion thereof) into the receiving tube portion 30. Extraction can include, by way of example and without limitation, chemicals, enzymes, shaking, centrifugation, and/or pneumatic transfer.

In one embodiment, a liquid carrier chamber 18 in the swab portion can contain a liquid carrier, which, for example, can be expressed manually or mechanically by squeezing the swab handle 16 (or a bladder therein). In one embodiment, an expressed liquid can travel through the lumen of the swab stem 12 and out the end of the swab 14, thereby eluting the specimen from the swab end 14. Alternatively, a liquid (e.g., an extraction buffer; 100 to 600 μl) can be introduced directly into the swab chamber and the device shaken to dislodge a specimen (or portion thereof) from the swab end.

In the embodiment shown in FIG. 1, a specimen eluted from a swab travels through a filter 26 before entering the lysis chamber 24. As used herein, a ‘filter’ generally has a pore size large enough that bacterial cells can pass through (e.g., greater than about 0.5 μm). A ‘filter’ as used herein can refer to a hydrophobic, partially-permeable material that requires force (e.g., pressure (e.g., hydrostatic) or centrifugal force) for liquid to pass through. A swab extraction device can be configured, for example, such that hydrostatic pressure can be generated by squeezing the swab handle (or a bladder associated therewith). In the embodiment shown in FIG. 1, the specimen leaves the swab end 14, passes through a filter 26 and enters the lysis chamber 24.

The specimen can be lysed (e.g., in a lysis chamber) by a number of different methods. For example, lysis can be initiated in the presence of silica or zirconium beads (e.g., 0.1 mm beads located within the lysis chamber) with a user applying mechanical or sonic vibration to lyse the cells. Alternatively, the lysis chamber can contain, for example, enzymes such as lysozyme, lysostaphin, and/or achromopeptidase, chemical reagents having a high pH (e.g., potassium hydroxide), and/or one or more detergents (e.g., sodium dodecyl sulfate). Following lysis, a specimen generally passes into a receiving tube portion. In the embodiment shown in FIG. 1, the lysed specimen passes through a filter 18 before entering the receiving tube portion.

One or more additives can be added to the specimen to, for example, stabilize the specimen or one or more components therein. An additive can be introduced into a specimen at any number of positions along the length of a swab extraction device. For example, an additive can be provided in a liquid carrier chamber 18 (e.g., in a liquid carrer), in a lysis reagent, or coated on the interior of the device (or a portion thereof). For example, EDTA (0.001 to 0.1 M) can be added to a specimen to help stabilize the extracted component (e.g., nucleic acids) or small amounts of protein or nucleic acid added can be added to the specimen to prevent binding of nucleic acids to the swab device.

A lysed specimen can be introduced into a receiving tube portion in any number of ways. In the embodiment shown in FIG. 1, the receiving tube portion 30 has a self-sealing septum 42 that can be penetrated by a needle 44 (e.g., a plastic needle) contained within the lower region of the swab transport/extraction portion. The extracted specimen then can be transferred into a receiving tube portion using, for example, centrifugal or hydrostatic pressure. Alternatively, a receiving tube portion can screw into the swab transport/extraction portion. A separate cap for the receiving tube portion would then be used to hold the specimen once the tube was removed from the transport/extraction component.

The swab extraction device disclosed herein is intended to provide ergonomic advantages over existing products.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A swab extraction device, wherein said device comprises:
a swab portion, wherein the swab portion comprises a handle, a swab stem, and a swab end;
a swab transport/extraction portion, wherein said swab transport /extraction portion comprises a swab chamber and a lysis chamber, wherein the swab chamber removeably houses the swab end, wherein the lysis chamber comprises a lysis reagent; and
a receiving tube portion, wherein the receiving tube portion is in fluid communication with the swab transport/extraction portion.

2. The device of claim 1, wherein said device further comprises at least one filter positioned between the swab chamber and the lysis chamber.

3. The device of claim 1, wherein said device further comprises at least one filter positioned between the lysis chamber and the receiving tube portion.

4. The device of claim 1, wherein said receiving tube portion is removeable from the device.

5. A method of using the swab extraction device of claim 1, comprising
obtaining a biological sample using said swab portion,
replacing said swab portion containing said biological sample to said swab chamber, and
transporting said device or said receiving tube portion to a laboratory for analysis.
